(19) United States
(12) Patent Application Publication
(10) Pub. No.: US 2024/0108881 A1
(43) Pub. Date: Apr. 4, 2024

(54) Flexible Electrode Arrays

(71) Applicant: NOVOCURE GMBH, Root D4 (CH)

(72) Inventors: Yoram Wasserman, Haifa (IL); Stas Obuchovsky, Haifa (IL); Nataliya Kuplennik, Haifa (IL); David Shapiro, Haifa (IL)

(21) Appl. No.: 18/476,433

(22) Filed: Sep. 28, 2023

Related U.S. Application Data
(60) Provisional application No. 63/411,299, filed on Sep. 29, 2022.

(51) Int. Cl. A61N 1/04

(52) U.S. Cl. CPC A61N 1/0496

(57) ABSTRACT

An apparatus has an electrode subassembly having a circuitry layer having a skin-facing inner side and an outer side. A plurality of electrode elements are disposed on the inner side of the circuitry layer and electrically coupled to the circuitry layer. Each electrode element of the plurality of electrode elements has an electrode edge. A layer of anisotropic material is electrically coupled to the plurality of electrode elements of the electrode subassembly. The layer of anisotropic material has a skin-facing surface and an opposing outwardly facing surface. The layer of anisotropic material has a peripheral outer edge. The peripheral outer edge of the layer of anisotropic material extends beyond the electrode edge of each respective electrode element of the plurality of electrode elements. A skin contact layer comprises a biocompatible conductive material. The skin contact layer is disposed on a skin-facing side of the layer of anisotropic material.

### CROSS-REFERENCE TO RELATED APPLICATIONS

This application claims priority to U.S. Provisional Patent Application No. 63/411,299, filed Sep. 29, 2022, the entirety of which is hereby incorporated by reference herein.

### BACKGROUND

Tumor Treating Fields (TTFields) therapy is a proven approach for treating tumors using alternating electric fields at frequencies between 50 kHz-1 MHz, more commonly, 100-500 kHz. In current commercial systems, the alternating electric fields are induced by electrode assemblies (e.g., arrays of capacitively coupled electrodes, also called transducer arrays) placed on opposite sides of a target region of the subject's body. When an AC voltage is applied between opposing electrode assemblies, an AC current is coupled through the electrode assemblies and into the subject's body. And higher currents are strongly correlated with higher efficacy of treatment.

### SUMMARY

TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a combination therapy with chemotherapy for malignant pleural mesothelioma (MPM), and may find use in treating tumors in other parts of the body. For applications targeting tumors in the torso, larger electrode arrays than currently used with the OPTUNE® system may be beneficial. What is needed is a larger area electrode array that is flexible enough to move with the body and be worn comfortably, while minimizing exposed printed circuit board (PCB) edges which can cause discomfort. Disclosed herein are flexible electrode arrays with minimal exposed PCB edges, which can be scaled to any area size including large areas suitable for torso applications.

Disclosed herein, in one aspect, an apparatus having an electrode subassembly having a circuitry layer and a plurality of electrode elements. The circuitry layer has a skin-facing inner side and an outer side. The plurality of electrode elements are disposed on the inner side of the circuitry layer and electrically coupled to the circuitry layer. Each electrode element of the plurality of electrode elements has an electrode edge. A layer of anisotropic material is electrically coupled to the plurality of electrode elements of the electrode subassembly. The layer of anisotropic material is disposed on an inner side of each of the plurality of electrode elements and has a skin-facing surface and an opposing outwardly facing surface. The layer of anisotropic material has a peripheral outer edge. The peripheral outer edge of the layer of anisotropic material extends beyond the electrode edge of each respective electrode element of the plurality of electrode elements. A skin contact layer comprises a biocompatible conductive material. The skin contact layer is disposed on a skin-facing side of the layer of anisotropic material.

In one aspect, an apparatus comprises an electrode subassembly having a circuitry layer and a plurality of electrode elements. The circuitry layer has a skin-facing inner side and an outer side and comprises a primary branch that extends along a first axis. The plurality of electrode elements are disposed on the inner side of the circuitry layer and electrically coupled to the circuitry layer. Each electrode element of the plurality of electrode elements has an electrode edge. A layer of anisotropic material is electrically coupled to the plurality of electrode elements of the electrode subassembly, the layer of anisotropic material having a skin-facing surface and an opposing outwardly facing surface. The layer of anisotropic material has a peripheral outer edge, wherein the peripheral outer edge of the layer of anisotropic material extends beyond the electrode edge of each respective electrode element of the plurality of electrode elements. A skin contact layer comprises a biocompatible conductive material. The skin contact layer is disposed on a skin-facing side of the layer of anisotropic material. The plurality of electrode elements include first and second electrode elements positioned on a first side of the primary branch and third and fourth electrode elements positioned on a second side of the primary branch. The second side is spaced from the first side along or parallel to a second axis that is perpendicular to the first axis. At least one of the first and second electrode elements positioned on the first side of the primary branch and at least one of the third and fourth electrode elements positioned on the second side of the primary branch are mechanically coupled to the primary branch in a manner that provides mechanical support and flexibility along both the first axis and the second axis.

Methods of using the apparatuses are also disclosed.

### BRIEF DESCRIPTION OF THE DRAWINGS

FIG. 1 is a top plan view of an electrode assembly in accordance with the present disclosure, with some layers shown as transparent in order to show underlying layers.

FIG. 2 is a cross-sectional view of the electrode assembly of FIG. 1, the cross-section taken in the plane 2-2′ shown in FIG. 1. Dimensions are not shown to scale.

FIG. 3 is a top plan view of an electrode assembly in accordance with the present disclosure, with some layers shown as transparent in order to show underlying layers.

FIG. 4 is a schematic diagram of a system for providing tumor-treating fields as disclosed herein.

Various embodiments are described in detail below with reference to the accompanying drawings, wherein like reference numerals represent like elements, and wherein descriptions of like elements may not be repeated for every embodiment, but may be considered to be the same if previously described herein.

### DESCRIPTION OF THE PREFERRED EMBODIMENTS

This application describes exemplary electrode assemblies that may be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” include plural referents unless the context clearly dictates otherwise. Thus, disclosure of “a layer” can represent disclosure of embodiments in which only a single layer is provided, as well as disclosure of embodiments in which a plurality of such layers are provided.

In the preceding and following description, the terms “front,” “inner,” and “skin-facing” are used interchangeably to refer to a face or surface of the disclosed electrode assemblies (or components thereof) that faces or is oriented toward the skin of a subject (or generally toward the body of a subject) when used as disclosed herein. Similarly, the terms “rear,” “upper,” “outer,” and “outwardly facing” are used interchangeably to refer to a face or surface of the disclosed electrode assemblies (or components thereof) that faces away from or is oriented away from the skin of a subject (or generally away from the body of the subject) when used as disclosed herein.

### Structure and Configuration of Apparatus

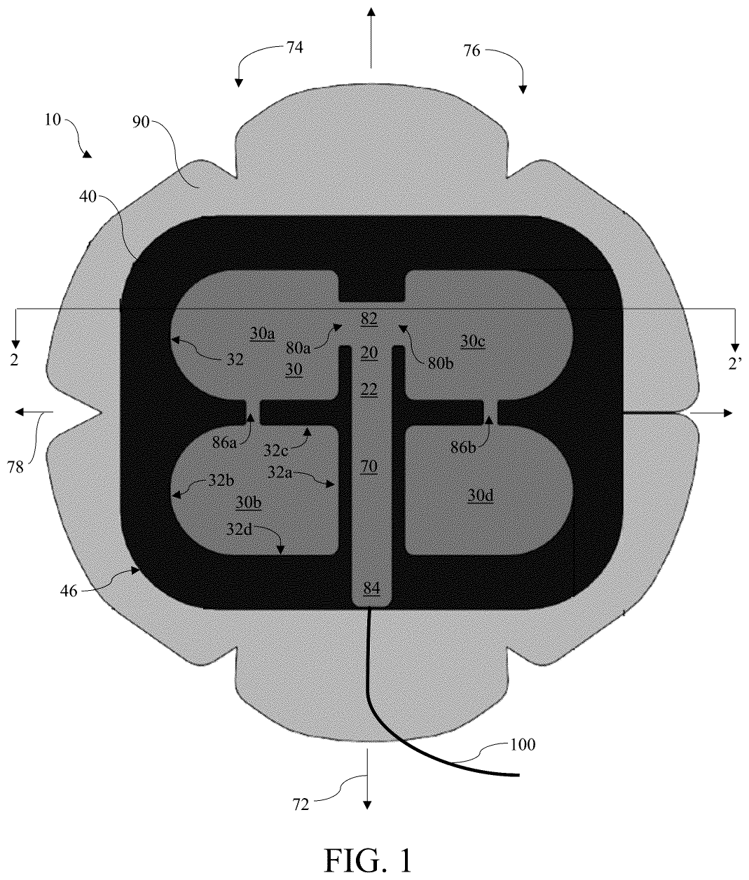

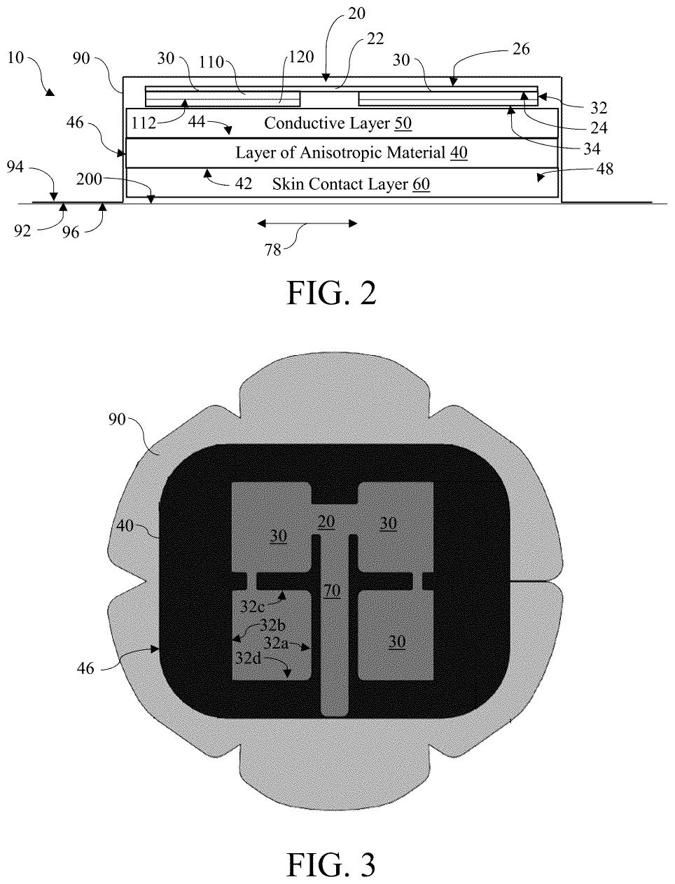

Referring to FIGS. 1 and 2, an apparatus 10 can comprise an electrode subassembly 20 having a circuitry layer 22 and a plurality of electrode elements 30 (e.g., electrode elements 30a-d in FIG. 1). The circuitry layer 22 has a skin-facing inner side 24 and an outer side 26. The plurality of electrode elements 30 can be disposed on the inner side 24 of the circuitry layer 22 and can be electrically coupled to the circuitry layer 22. Each electrode element 30 of the plurality of electrode elements can have an electrode edge 32. The circuitry layer 22 can optionally comprise (or be) a printed circuit board (PCB).

A layer of anisotropic material 40 can be electrically coupled to the plurality of electrode elements 30 of the electrode subassembly 20. The layer of anisotropic material 40 can have a skin-facing surface 42 and an opposing outwardly facing surface 44. The layer of anisotropic material 40 can have a peripheral outer edge 46. The peripheral outer edge 46 of the layer of anisotropic material 40 can extend beyond the electrode edge 32 of each respective electrode element 30 of the plurality of electrode elements. In an embodiment, the peripheral outer edge 46 of the layer of anisotropic material 40 can extend beyond the electrode edge 32 of each respective electrode element 30 of the plurality of electrode elements by at least 1 mm, or at least: 2 mm, 3 mm, 4 mm, 5 mm, 6 mm, 7 mm, 8 mm, 9 mm, 10 mm, 15 mm, 20 mm, 30 mm, 40 mm, or 50 mm.

The apparatus 10 can further comprise a skin contact layer 60 comprising a biocompatible conductive material. The skin contact layer 60 can be disposed on a skin-facing side 48 of the layer of anisotropic material 40, and, when the apparatus is in use treating a patient, the skin contact layer 60 can be in contact with the subject's skin 200. Optionally, the skin contact layer 60 can be disposed against the skin-facing surface 42 of the layer of anisotropic material 40. In some aspects, the skin contact layer 60 can be or comprise hydrogel. In other aspects, the skin contact layer 60 can be or comprise a conductive adhesive composite.

In some aspects, the circuitry layer 22 can comprise a primary branch 70 that extends along a first axis 72. The plurality of electrode elements 30 can comprise at least one electrode element (e.g., electrode elements 30a,b) positioned on a first side 74 of the primary branch 70 and at least one electrode element (e.g., electrode element 30c,d) positioned on a second side 76 of the primary branch. The second side 76 can be spaced from the first side 74 along or parallel to a second axis 78 that is perpendicular to the first axis 72.

In various aspects, at least one electrode element 30 positioned on the first side 74 of the primary branch 70 (e.g., first and/or second electrode elements 30a,b) and at least one electrode element 30 positioned on the second side 76 of the primary branch 70 (e.g., third and/or fourth electrode elements 30c,d) can be mechanically coupled to the primary branch in a manner that provides mechanical support and flexibility along both the first axis 72 and the second axis 78.

In some aspects, the one or more of the electrode elements 30 positioned on the first side 74 of the primary branch 70 can comprise first and second electrode elements 30a,b positioned on the first side of the primary branch. In further aspects, the one or more of the electrode elements 30 positioned on the second side 76 of the primary branch 70 can comprise third and fourth electrode elements 30c,d positioned on the second side 76 of the primary branch 70.

The circuitry layer 22 can comprise a first secondary branch 80a that extends away from a first end portion 82 of the primary branch 70 in a first direction along or parallel to the second axis 78. A second secondary branch 80b can extend away from the first end portion 82 of the primary branch 70 in a second direction along or parallel to the second axis 78 that is opposite the first direction. The first secondary branch 80a can electrically and mechanically couple the first electrode element 30a to the primary branch 70. The second secondary branch 80b can electrically and mechanically couple the third electrode element 30c to the primary branch 70.

The circuitry layer 22 can further comprise a first tertiary branch 86a that extends away from the first electrode element 30a along or parallel to the first axis 72 in a direction toward a second end portion 84 of the primary branch 70 and a second tertiary branch 86b that extends away from the third electrode element 30c along or parallel to the first axis 72 in the direction toward the second end portion 84 of the primary branch 70. The first tertiary branch 86a can electrically and mechanically couple the second electrode element 30b to the first secondary branch 80a, and the second tertiary branch 86b can electrically and mechanically couple the fourth electrode element 30d to the second secondary branch 80b.

The respective electrode edges 32 of the second and fourth electrode elements 30b,d can be spaced from the primary branch 70 along or parallel to the second axis 78.

In some optional aspects, each electrode element 30 of the plurality of electrode elements can comprise a first end edge 32a that is parallel or substantially parallel to the first axis 72. The first end edge 32a of each electrode element 30 can face the primary branch 70.

In further optional aspects, each electrode element 30 of the plurality of electrode elements can further comprise an opposing second end edge 32b that is rounded and that faces a periphery 46 of the layer of anisotropic material 40.

Each electrode element 30 of the plurality of electrode elements can further comprise first and second side edges 32c,d that extend between the first and second end edges 32a,b of the electrode element.

In some optional aspects, and with reference to FIG. 3, each electrode element 30 of the plurality of electrode elements can comprise a second end edge 32b that is parallel or substantially parallel to the first axis 72 and that faces a periphery 46 of the layer of anisotropic material 40.

In some aspects, at least one electrode element 30 of the plurality of electrode elements can have a circular or oval shape. That is, the electrode edge 32 of at least one electrode element can be circular or oval.

The primary branch 70 of the circuitry layer 22 can have a length. In some optional aspects, the primary branch 70 of the circuitry layer 22 (e.g., the PCB) can be wrapped in a polymeric protective covering along a portion of, most of, substantially all of, or all of, the length of the primary branch. The polymeric protective covering can cover the edges of the primary branch 70 (e.g., PCB edges). In this way, the polymeric protective coating can minimize any discomfort from the PCB edges contacting the subject's skin 200. Further optionally, fewer electrode elements 30 translates to fewer areas of exposed PCB connecting the electrode elements 30 (and fewer exposed PCB edges). For example, FIG. 1 and FIG. 3 illustrate electrode arrays having just 4 electrode elements 30, thereby minimizing the number of PCB edges, while allowing flexibility in directions moving around, for example, a first axis 72 and a second axis 78.

In some optional aspects, the apparatus 10 can comprise a conductive layer 50 positioned between a respective skin-facing surface 34 of each of the plurality of electrode elements 30 and the outwardly facing surface 44 of the layer of anisotropic material 40. In further optional aspects, the conductive layer 50 can be, or comprise, a layer of hydrogel. In further optional aspects, the conductive layer 50 can be, or comprise, a layer of conductive adhesive composite. The conductive layer 50 (e.g., layer of hydrogel or layer of conductive adhesive composite) can be configured to facilitate electrical contact between the plurality of electrode elements 30 and the outwardly facing surface 44 of the layer of anisotropic material 40. In some optional aspects, the conductive layer 50 can be omitted from the apparatus 10.

Optionally, the apparatus 10 can comprise a covering layer 90 having an inner side 92 and an outer side 94. The inner side 92 can be disposed on the outer side of the circuitry layer 22. Portions of the covering layer 90 can extend beyond the electrode edge 32 of each of the electrode elements 30 and beyond a periphery of (e.g., the peripheral outer edge 46 of) the layer of anisotropic material 40 to define at least one attachment surface 96.

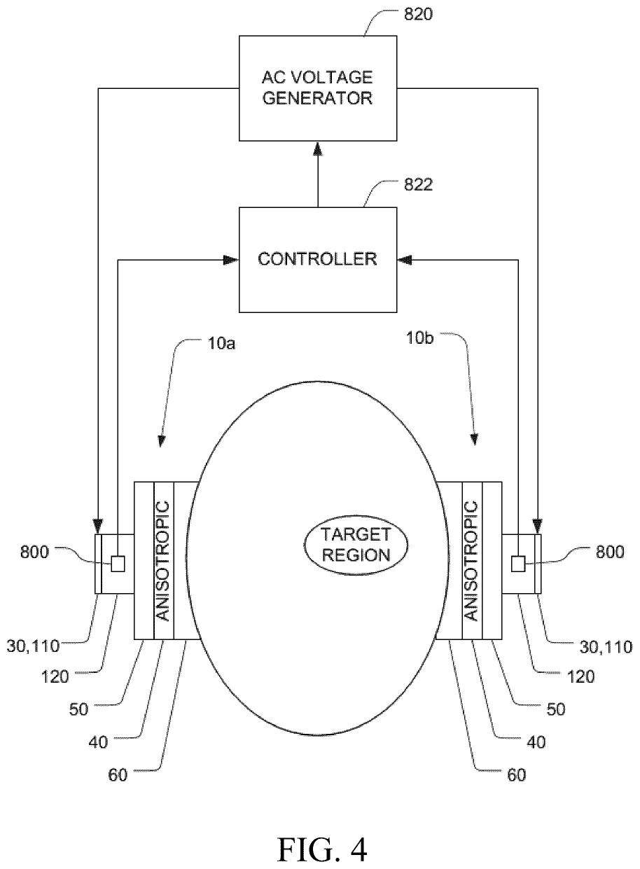

The apparatus 10 can further comprise a single wire 100 (FIG. 1) that is configured to electrically couple the electrode subassembly to a current source (e.g., an AC voltage generator 820 (FIG. 4)).

The electrode subassembly 20 can have a total areal footprint, and the layer of anisotropic material 40 can have a total areal footprint. In some optional aspects, a ratio of the total areal footprint of the electrode subassembly 20 to the total areal footprint of the layer of anisotropic material 40 can be from 20% to 95%, such as, for example, 25% to 90%, or 25% to 85%; and can range from as low as 20%, or 25%, or 30%, or 40%, or 50%, or 60%, or 70%, and up to as high as 50%, or 60%, or 70%, or 80%, or 85%, or 90%, or 95%, in any combination of endpoints in the range.

In some optional aspects, each electrode element 30 can comprise a metallic layer 110 having a skin-facing surface 112 and a layer of dielectric material 120. The layer of dielectric material 120 can be disposed on the skin-facing side of the metallic layer 110, such as on the skin-facing surface 112 of the metallic layer 110, and can be electrically coupled to the outwardly facing surface 44 of the layer of anisotropic material 40. In some aspects, the dielectric material 120 can comprise ceramic material. In other aspects, the dielectric material 120 can comprise polymer film. In exemplary aspects, the dielectric material 120 can have a dielectric constant ranging from 10 to 50,000. In some embodiments, the layer of dielectric material 120 comprises a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. These embodiments are particularly advantageous because the dielectric constant of these materials is on the order of 40. In some embodiments, the polymer layer can be poly(vinylidene fluoride-trifluoroethylene-chlorotrifluoroethylene-chlorofluoroethylene) or “Poly(VDF-TrFE-CTFE-CFE).” In some embodiments, the layer of dielectric material 70 comprises a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

In alternative aspects, the electrode elements 30 do not comprise a dielectric material.

In some optional aspects, the anisotropic material 40 can comprise graphite. In some optional aspects, the graphite can comprise synthetic graphite. The layer of anisotropic material can be, or can comprise, a layer of pyrolytic graphite, graphitized polymer film, or graphite foil made from compressed high purity exfoliated mineral graphite. Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied by MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan. In alternative embodiments, conductive anisotropic materials other than graphite may be used instead of graphite.

In some aspects, the layer of anisotropic material 40 has a first thermal conductivity in a direction that is perpendicular to a plane of the layer. The thermal conductivity of the layer of anisotropic material 40 in directions that are parallel to the plane of the layer of anisotropic material can be more than two times higher than the first thermal conductivity. For example, in some aspects, the thermal conductivity of the layer of anisotropic material 40 in directions that are parallel to the plane of the layer of anisotropic material can be more than three times higher, more than four times higher, or more five than the first thermal conductivity. In some aspects, the thermal conductivity in the parallel directions is more than ten times higher than the first thermal conductivity. In various aspects, the thermal conductivity of the layer of isotropic material 40 in directions that are parallel to the plane of the layer of anisotropic material can be more than: 1.5 times, 2 times, 3 times, 5 times, 10 times, 20 times, 100 times, 200 times, or even more than 1,000 times higher than the first thermal conductivity. The use of a layer of anisotropic material 40 in the electrode array facilitates the current entering the body over a larger area, and may be advantageous in larger arrays, such as those intended for use on the torso.

The layer of anisotropic material 40 can have a first resistance in a direction that is perpendicular to a plane of the layer. In some optional aspects, resistance of the layer in directions that are parallel to the plane of the layer is less than half the first resistance. In exemplary aspects, the resistance of the layer of anisotropic material 40 in directions that are parallel to the plane of the layer can be less than 10% of the first resistance. In exemplary aspects, the resistance of the layer of anisotropic material 40 in directions that are parallel to the plane of the layer can be less than 75%, 50%, 40%, 30%, 20%, 10%, 5%, 1%, 0.5%, or even less than 0.1% of the first resistance.

In some optional aspects, the layer of anisotropic material 40 can be omitted from the apparatus 10.

In some aspects, the skin contact layer 60 may be a layer of hydrogel. In some aspects, the skin contact layer 60 may be a layer of conductive adhesive composite. In some aspects, the conductive layer 50 may be a layer of hydrogel. In some aspects, the conductive layer 50 may be a layer of conductive adhesive composite.

In exemplary aspects, the conductive adhesive composite of any of the layers of the apparatus 10 (e.g., the skin contact layer 60 and/or the conductive layer 50) can comprise a dielectric material and conductive particles dispersed within the dielectric material. In some embodiments, at least a portion of the conductive particles define a conductive pathway through a thickness of the conductive adhesive composite. It is contemplated that the conductive particles can be aligned in response to application of an electric field such that the conductive particles undergo electrophoresis. In some aspects, the dielectric material of the conductive adhesive composite can be a polymeric adhesive. Optionally, in these aspects, the polymeric adhesive can be an acrylic adhesive or a silicone adhesive. In some aspects, the conductive particles can comprise carbon. Optionally, in these aspects, the conductive particles can comprise graphite powder. Additionally, or alternatively, the conductive particles can comprise carbon flakes. Additionally, or alternatively, the conductive particles can comprise carbon granules. Additionally, or alternatively, the conductive particles can comprise carbon fibers. Additionally, or alternatively, the conductive particles can comprise carbon nanotubes or carbon nanowires. Additionally, or alternatively, the conductive particles can comprise carbon black powder. In further aspects, the conductive adhesive composite further comprises a polar material (e.g., a polar salt). The polar salt may be a quaternary ammonium salt, such as a tetra alkyl ammonium salt. Exemplary conductive adhesive composites, as well as methods for making such conductive adhesive composites, are disclosed in U.S. Pat. No. 8,673,184 and U.S. Pat. No. 9,947,432, which are incorporated herein by reference for all purposes. In exemplary aspects, the conductive adhesive composite can be a dry carbon/salt adhesive.

In exemplary aspects, the conductive layer 50 or the skin contact layer 60 can comprise a conductive adhesive composite provided by ADHESIVE RESEARCH, such as ARcare® 8006 electrically conductive adhesive composition manufactured and sold by Adhesives Research, Inc. (Glen Rock, PA, USA). In other optional aspects, the conductive layer 50 and/or the skin contact layer 60 can comprise carbon fibers or nanowires. For example, in exemplary aspects, the conductive layer 50 and/or the skin contact layer 60 can comprise a dry carbon/salt adhesive, such as the developmental product FLX068983—FLEXcon® OMNI-WAVE™ TT 200 BLACK H-502 150 POLY H-9 44PP-8 from FLEXcon, Spencer, MA, USA, or other such OMNI-WAVE products from FLEXcon.

In various aspects, the conductive layer 50 can have a thickness from about 25 μm to about 150 μm. In various aspects, the skin contact layer 60 can have a thickness from about 25 μm to about 150 μm.

### EXEMPLARY METHOD OF USE OF APPARATUS

Referring to FIG. 4, a method can comprise applying an electrical field using at least one electrode subassembly 20 of the apparatus 10, where components of apparatus 10 can be as described above (and as labelled in FIGS. 1-3). For example, at least first and second apparatuses 10a,b can be positioned on a body of a subject or within the body of a subject. The skin contact layer 60 of each of the first and second apparatuses 10a,b can contact skin 200 (FIG. 2) of the subject. An alternating voltage can be applied between the first apparatus 10a and the second apparatus 10b, thereby generating an electric field.

The alternating voltage between the first apparatus 10a and the second apparatus 10b can be applied by an AC voltage generator 820. In some embodiments, the frequency of the alternating voltage is between 50 kHz and 1 MHz, or between 100 kHz and 500 kHz. In the illustrated example, the AC voltage generator is controlled by a controller 822. The controller 822 may use temperature measurements to control the amplitude of the current to be delivered via the first and second apparatus 10a,b in order to maintain temperatures below a safety threshold (e.g., 41° C.). This may be accomplished, for example, by measuring a first temperature of a first electrode element 30, measuring a second temperature of a second electrode element 30, and controlling the applying of the alternating voltage based on the first temperature and the second temperature, as described below.

FIG. 4 depicts one example of hardware that is suitable for this purpose. More specifically, temperature sensors 800 (e.g., thermistors) are positioned in thermal contact with respective electrode elements 30 (for example, dielectric material 120/layer of metal 110) within each of the apparatuses 10a,b. The temperature sensors 800 measure respective first and second temperatures (e.g., at first and second electrode elements in the first electrode assembly and second electrode assembly, respectively), and the controller 822 controls the output of the AC voltage generator 820 based on these temperatures.

Optionally, prior to applying the electrical field, a shape and/or size of the apparatus 10 can be adjusted by cutting through a peripheral portion of the layer of anisotropic material 40 that is positioned beyond the electrode edge 32 of each respective electrode element 30 of the plurality of electrode elements.

### EXEMPLARY EMBODIMENTS

Referring to FIGS. 1-2, an apparatus 10 can comprise an electrode subassembly 20 having a circuitry layer 22, the circuitry layer having a skin-facing inner side 24 and an outer side 26. The circuitry layer 22 can comprise a primary branch 70 that extends along a first axis 72. A plurality of electrode elements 30 can be disposed on the inner side 24 of the circuitry layer 22 and can be electrically coupled to the circuitry layer 22. Each electrode element 30 of the plurality of electrode elements can have an electrode edge 32.

A layer of anisotropic material 40 can be electrically coupled to the plurality of electrode elements 30 of the electrode subassembly 20. The layer of anisotropic material 40 can have a skin-facing surface 42 and an opposing outwardly facing surface 44. The layer of anisotropic material 40 can have a peripheral outer edge 46. In some optional aspects, the peripheral outer edge 46 of the layer of anisotropic material 40 can extend beyond the electrode edge 32 of each respective electrode element 30 of the plurality of electrode elements. In some optional aspects, the peripheral outer edge 46 of the layer of anisotropic material 40 can extend beyond the electrode edge 32 of each respective electrode element 30 of the plurality of electrode elements by at least 1 mm, or at least: 2 mm, 3 mm, 4 mm, 5 mm, 6 mm, 7 mm, 8 mm, 9 mm, 10 mm, 15 mm, 20 mm, 30 mm, 40 mm, or 50 mm.

The apparatus 10 can further comprise a skin contact layer 60 comprising a biocompatible conductive material. The skin contact layer 60 can be disposed on a skin-facing side 48 of the layer of anisotropic material 40. Optionally, the skin contact layer 60 can be disposed against the skin-facing surface 42 of the layer of anisotropic material 40. In some aspects, the skin contact layer can be hydrogel. In other aspects, the skin contact layer can be a conductive adhesive composite.

In some optional aspects, the apparatus 10 can further comprise the same components as described earlier for apparatus 10. For example, the apparatus 10 can comprise a conductive layer 50 positioned between a respective skin-facing surface 34 of each of the plurality of electrode elements 30 and the outwardly facing surface 44 of the layer of anisotropic material 40. In further optional aspects, the conductive layer 50 can be, or comprise, a layer of hydrogel. In further optional aspects, the conductive layer 50 can be, or comprise, a layer of conductive adhesive composite.

The plurality of electrode elements comprise first and second electrode elements 30a,b positioned on a first side 74 of the primary branch 70 and third and fourth electrode elements 30c,d positioned on a second side 76 of the primary branch 70. The second side 76 can be spaced from the first side 74 along or parallel to a second axis 78 that is perpendicular to the first axis 72.

In various aspects, at least one of the first and second electrode elements 30a,b positioned on the first side 74 of the primary branch 70 and at least one of the third and fourth electrode elements 30c,d positioned on the second side 76 of the primary branch 70 can be mechanically coupled to the primary branch in a manner that provides mechanical support and flexibility along both the first axis 72 and the second axis 78.

The electrode subassembly 20 can have a total areal footprint, and the layer of anisotropic material 40 can have a total areal footprint. In some optional aspects, a ratio of the total areal footprint of the electrode subassembly 20 to the total areal footprint of the layer of anisotropic material 40 can be from 20% to 95%, such as, for example, 25% to 90%, or 25% to 85%; and can range from as low as 20%, or 25%, or 30%, or 40%, or 50%, or 60%, or 70%, and up to as high as 50%, or 60%, or 70%, or 80%, or 85%, or 90%, or 95%, in any combination of endpoints in the range.

The exemplary embodiments can be used in accordance with methods disclosed herein.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1. An apparatus comprising:
an electrode subassembly having:
   a circuitry layer having a skin-facing inner side and an outer side; and
   a plurality of electrode elements disposed on the inner side of the circuitry layer and electrically coupled to the circuitry layer, wherein each electrode element of the plurality of electrode elements has an electrode edge and an inner side;
a layer of anisotropic material electrically coupled to the plurality of electrode elements of the electrode subassembly, the layer of anisotropic material disposed on the inner side of each electrode element of the plurality of electrode elements and having a skin-facing surface and an opposing outwardly facing surface, the layer of anisotropic material having a peripheral outer edge, wherein the peripheral outer edge of the layer of anisotropic material extends beyond the electrode edge of each respective electrode element of the plurality of electrode elements; and
a skin contact layer comprising a biocompatible conductive material, wherein the skin contact layer is disposed on a skin-facing side of the layer of anisotropic material.

Aspect 2. The apparatus of aspect 1, wherein the circuitry layer comprises a primary branch that extends along a first axis, wherein the plurality of electrode elements comprise:
at least one electrode element positioned on a first side of the primary branch; and
at least one electrode element positioned on a second side of the primary branch, wherein the second side is spaced from the first side along a second axis that is perpendicular to the first axis,
wherein at least one of the at least one electrode element positioned on the first side of the primary branch and at least one of the at least one electrode element positioned on the second side of the primary branch are mechanically coupled to the primary branch in a manner that provides mechanical support and flexibility along both the first axis and the second axis.

Aspect 3. The apparatus of aspect 2, wherein the at least one electrode element positioned on the first side of the primary branch comprises first and second electrode elements positioned on the first side of the primary branch, and wherein the at least one electrode element positioned on the second side of the primary branch comprises third and fourth electrode elements positioned on the second side of the primary branch.

Aspect 4. The apparatus of aspect 3, wherein the circuitry layer comprises:
a first secondary branch that extends away from a first end portion of the primary branch in a first direction along or parallel to the second axis; and
a second secondary branch that extends away from the first end portion of the primary branch in a second direction along or parallel to the second axis that is opposite the first direction,
wherein the first secondary branch electrically and mechanically couples the first electrode element to the primary branch, and wherein the second secondary branch electrically and mechanically couples the third electrode element to the primary branch.

Aspect 5. The apparatus of aspect 4, wherein the circuitry layer further comprises:
a first tertiary branch that extends away from the first electrode element along or parallel to the first axis in a direction toward a second end portion of the primary branch; and
a second tertiary branch that extends away from the third electrode element along or parallel to the first axis in the direction toward the second end portion of the primary branch,
wherein the first tertiary branch electrically and mechanically couples the second electrode element to the first secondary branch, and wherein the second tertiary branch electrically and mechanically couples the fourth electrode element to the second secondary branch.

Aspect 6. The apparatus of aspect 5, wherein the respective electrode edges of the second and fourth electrode elements are spaced from the primary branch along or parallel to the second axis.

Aspect 7. The apparatus of any one of aspects 2-6, wherein each electrode element of the plurality of electrode elements comprises a first end edge that is parallel or substantially parallel to the first axis and that faces the primary branch.

Aspect 8. The apparatus of aspect 7, wherein each electrode element of the plurality of electrode elements further comprises an opposing second end edge that is rounded and that faces the peripheral outer edge of the layer of anisotropic material.

Aspect 9. The apparatus of aspect 8, wherein each electrode element of the plurality of electrode elements further comprises first and second side edges that extend between the first and second end edges of the electrode element.

Aspect 10. The apparatus of aspect 7, wherein each electrode element of the plurality of electrode elements further comprises an opposing second end edge that is parallel or substantially parallel to the first axis and that faces the peripheral outer edge of the layer of anisotropic material.

Aspect 11. The apparatus of any one of aspects 2-6, wherein at least one electrode element of the plurality of electrode elements has a circular or oval shape.

Aspect 12. The apparatus of any one of the preceding aspects, further comprising a layer of conductive adhesive composite positioned between a skin-facing surface of the plurality of electrode elements of the electrode subassembly and the outwardly facing surface of the layer of anisotropic material, wherein the layer of conductive adhesive composite is configured to facilitate electrical contact between the plurality of electrode elements and the outwardly facing surface of the layer of anisotropic material.

Aspect 13. The apparatus of any one of the preceding aspects, further comprising a covering layer having an inner side and an outer side, wherein the inner side is disposed on the outer side of the circuitry layer, wherein portions of the covering layer extend beyond the electrode edge of each of the electrode elements and beyond the peripheral outer edge of the layer of anisotropic material to define at least one attachment surface.

Aspect 14. The apparatus of any one of the preceding aspects, further comprising a single wire that is configured to electrically couple the electrode subassembly to a current source.

Aspect 15. The apparatus of any one of the preceding aspects, wherein the electrode subassembly has a total areal footprint, wherein the layer of anisotropic material has a total areal footprint, and wherein a ratio of the total areal footprint of the electrode subassembly to the total areal footprint of the layer of anisotropic material is from 20% to 95%.

Aspect 16. The apparatus of any one of the preceding aspects, wherein each electrode element comprises:
a metallic layer having a skin-facing side and a skin-facing surface; and
a layer of dielectric material, wherein the layer of dielectric material is disposed on the skin-facing side of the metallic layer and is electrically coupled to both of the metallic layer and the outwardly facing surface of the layer of anisotropic material.

Aspect 17. The apparatus of aspect 16, wherein the layer of dielectric material comprises a ceramic material.

Aspect 18. The apparatus of aspect 16, wherein the layer of dielectric material is a polymer film.

Aspect 19. The apparatus of any one of the preceding aspects, wherein the anisotropic material comprises graphite.

Aspect 20. The apparatus of any one of the preceding aspects, wherein the skin-contact layer is a hydrogel.

Aspect 21. The apparatus of any one of the preceding aspects, wherein the skin-contact layer is a conductive adhesive composite.

Aspect 22. The apparatus of any one of the preceding aspects, wherein the skin-contact layer is disposed on the skin-facing surface of the layer of anisotropic material.

Aspect 23. The apparatus of any one of the preceding aspects, wherein the anisotropic material comprises graphite, and wherein the peripheral outer edge of the layer of anisotropic material extends beyond the electrode edge of each respective electrode element of the plurality of electrode elements by at least 1 mm.

Aspect 24. A method comprising:
applying an electrical field using the at least one electrode subassembly of the apparatus of any one of the preceding aspects.

Aspect 25. The method of aspect 24, further comprising, prior to applying the electrical field, adjusting a shape and/or size of the apparatus by cutting through a peripheral portion of the layer of anisotropic material that is positioned beyond the electrode edge of each respective electrode element of the plurality of electrode elements.

Aspect 26. An apparatus comprising:
an electrode subassembly having:
   a circuitry layer having a skin-facing inner side and an outer side, wherein the circuitry layer comprises a primary branch that extends along a first axis; and
   a plurality of electrode elements disposed on the inner side of the circuitry layer and electrically coupled to the circuitry layer, wherein each electrode element of the plurality of electrode elements has an electrode edge;
a layer of anisotropic material electrically coupled to the plurality of electrode elements of the electrode subassembly, the layer of anisotropic material having a skin-facing surface and an opposing outwardly facing surface, the layer of anisotropic material having a peripheral outer edge, wherein the peripheral outer edge of the layer of anisotropic material extends beyond the electrode edge of each respective electrode element of the plurality of electrode elements; and
a skin contact layer comprising a biocompatible conductive material, wherein the skin contact layer is disposed on a skin-facing side of the layer of anisotropic material,
wherein the plurality of electrode elements comprise:
   first and second electrode elements positioned on a first side of the primary branch; and
   third and fourth electrode elements positioned on a second side of the primary branch, wherein the second side is spaced from the first side along or parallel to a second axis that is perpendicular to the first axis,
wherein at least one of the first and second electrode elements positioned on the first side of the primary branch and at least one of the third and fourth electrode elements positioned on the second side of the primary branch are mechanically coupled to the primary branch in a manner that provides mechanical support and flexibility along both the first axis and the second axis.

Aspect 27. The apparatus of aspect 26, wherein the peripheral outer edge of the layer of anisotropic material extends beyond the electrode edge of each respective electrode element of the plurality of electrode elements by at least 1 mm.

Aspect 28. The apparatus of aspect 26 or aspect 27, wherein the electrode subassembly has a total areal footprint, wherein the layer of anisotropic material has a total areal footprint, and wherein a ratio of the total areal footprint of the electrode subassembly to the total areal footprint of the layer of anisotropic material is from 20% to 95%.

Aspect 29. A method comprising:
applying an electrical field using the electrode subassembly of the apparatus of any one of aspects 26-28.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus comprising:
an electrode subassembly having:
a circuitry layer having a skin-facing inner side and an outer side; and
a plurality of electrode elements disposed on the inner side of the circuitry layer and electrically coupled to the circuitry layer, wherein each electrode element of the plurality of electrode elements has an electrode edge and an inner side;
a layer of anisotropic material electrically coupled to the plurality of electrode elements of the electrode subassembly, the layer of anisotropic material disposed on the inner side of each electrode element of the plurality of electrode elements and having a skin-facing surface and an opposing outwardly facing surface, the layer of anisotropic material having a peripheral outer edge, wherein the peripheral outer edge of the layer of anisotropic material extends beyond the electrode edge of each respective electrode element of the plurality of electrode elements; and
a skin contact layer comprising a biocompatible conductive material, wherein the skin contact layer is disposed on a skin-facing side of the layer of anisotropic material.

2. The apparatus of claim 1, wherein the circuitry layer comprises a primary branch that extends along a first axis, wherein the plurality of electrode elements comprise:
at least one electrode element positioned on a first side of the primary branch; and
at least one electrode element positioned on a second side of the primary branch, wherein the second side is spaced from the first side along a second axis that is perpendicular to the first axis,
wherein at least one of the at least one electrode element positioned on the first side of the primary branch and at least one of the at least one electrode element positioned on the second side of the primary branch are mechanically coupled to the primary branch in a manner that provides mechanical support and flexibility along both the first axis and the second axis.

3. The apparatus of claim 2, wherein the at least one electrode element positioned on the first side of the primary branch comprises first and second electrode elements positioned on the first side of the primary branch, and wherein the at least one electrode element positioned on the second side of the primary branch comprises third and fourth electrode elements positioned on the second side of the primary branch.

4. The apparatus of claim 3, wherein the circuitry layer comprises:
a first secondary branch that extends away from a first end portion of the primary branch in a first direction along or parallel to the second axis; and
a second secondary branch that extends away from the first end portion of the primary branch in a second direction along or parallel to the second axis that is opposite the first direction,
wherein the first secondary branch electrically and mechanically couples the first electrode element to the primary branch, and wherein the second secondary branch electrically and mechanically couples the third electrode element to the primary branch.

5. The apparatus of claim 4, wherein the circuitry layer further comprises:
a first tertiary branch that extends away from the first electrode element along or parallel to the first axis in a direction toward a second end portion of the primary branch; and
a second tertiary branch that extends away from the third electrode element along or parallel to the first axis in the direction toward the second end portion of the primary branch,
wherein the first tertiary branch electrically and mechanically couples the second electrode element to the first secondary branch, and wherein the second tertiary branch electrically and mechanically couples the fourth electrode element to the second secondary branch.

6. The apparatus of claim 5, wherein the respective electrode edges of the second and fourth electrode elements are spaced from the primary branch along or parallel to the second axis.

7. The apparatus of claim 2, wherein each electrode element of the plurality of electrode elements comprises a first end edge that is parallel or substantially parallel to the first axis and that faces the primary branch.

8. The apparatus of claim 7, wherein each electrode element of the plurality of electrode elements further comprises an opposing second end edge that is rounded and that faces the peripheral outer edge of the layer of anisotropic material.

9. The apparatus of claim 8, wherein each electrode element of the plurality of electrode elements further comprises first and second side edges that extend between the first and second end edges of the electrode element.

10. The apparatus of claim 7, wherein each electrode element of the plurality of electrode elements further comprises an opposing second end edge that is parallel or substantially parallel to the first axis and that faces the peripheral outer edge of the layer of anisotropic material.

11. The apparatus of claim 1, further comprising a layer of conductive adhesive composite positioned between a skin-facing surface of the plurality of electrode elements of the electrode subassembly and the outwardly facing surface of the layer of anisotropic material, wherein the layer of conductive adhesive composite is configured to facilitate electrical contact between the plurality of electrode elements and the outwardly facing surface of the layer of anisotropic material.

12. The apparatus of claim 1, further comprising a covering layer having an inner side and an outer side, wherein the inner side is disposed on the outer side of the circuitry layer, wherein portions of the covering layer extend beyond the electrode edge of each of the electrode elements and beyond the peripheral outer edge of the layer of anisotropic material to define at least one attachment surface.

13. The apparatus of claim 1, wherein the electrode subassembly has a total areal footprint, wherein the layer of anisotropic material has a total areal footprint, and wherein a ratio of the total areal footprint of the electrode subassembly to the total areal footprint of the layer of anisotropic material is from 20% to 95%.

14. The apparatus of claim 1, wherein each electrode element comprises:
a metallic layer having a skin-facing side and a skin-facing surface; and
a layer of dielectric material, wherein the layer of dielectric material is disposed on the skin-facing side of the metallic layer and is electrically coupled to both of the metallic layer and the outwardly facing surface of the layer of anisotropic material.

15. The apparatus of claim 14, wherein the layer of dielectric material comprises a ceramic material or a polymer film.

16. The apparatus of claim 1, wherein the skin-contact layer is a conductive adhesive composite.

17. The apparatus of claim 1, wherein the anisotropic material comprises graphite, and wherein the peripheral outer edge of the layer of anisotropic material extends beyond the electrode edge of each respective electrode element of the plurality of electrode elements by at least 1 mm.

18. An apparatus comprising:
an electrode subassembly having:
a circuitry layer having a skin-facing inner side and an outer side, wherein the circuitry layer comprises a primary branch that extends along a first axis; and
a plurality of electrode elements disposed on the inner side of the circuitry layer and electrically coupled to the circuitry layer, wherein each electrode element of the plurality of electrode elements has an electrode edge;
a layer of anisotropic material electrically coupled to the plurality of electrode elements of the electrode subassembly, the layer of anisotropic material having a skin-facing surface and an opposing outwardly facing surface, the layer of anisotropic material having a peripheral outer edge, wherein the peripheral outer edge of the layer of anisotropic material extends beyond the electrode edge of each respective electrode element of the plurality of electrode elements; and
a skin contact layer comprising a biocompatible conductive material, wherein the skin contact layer is disposed on a skin-facing side of the layer of anisotropic material,
wherein the plurality of electrode elements comprise:
first and second electrode elements positioned on a first side of the primary branch; and
third and fourth electrode elements positioned on a second side of the primary branch, wherein the second side is spaced from the first side along or parallel to a second axis that is perpendicular to the first axis,
wherein at least one of the first and second electrode elements positioned on the first side of the primary branch and at least one of the third and fourth electrode elements positioned on the second side of the primary branch are mechanically coupled to the primary branch in a manner that provides mechanical support and flexibility along both the first axis and the second axis.

19. The apparatus of claim 18, wherein the peripheral outer edge of the layer of anisotropic material extends beyond the electrode edge of each respective electrode element of the plurality of electrode elements by at least 1 mm.

20. The apparatus of claim 18, wherein the electrode subassembly has a total areal footprint, wherein the layer of anisotropic material has a total areal footprint, and wherein a ratio of the total areal footprint of the electrode subassembly to the total areal footprint of the layer of anisotropic material is from 20% to 95%.
